(19) United States
(12) Patent Application Publication
(10) Pub. No.: US 2006/0110787 A1
(43) Pub. Date: May 25, 2006

(54) Use of fluorine-18-labeled fluoroquinolone antibiotics for diagnosing and monitoring bacterial infection

(75) Inventor: Ronald C. Walker, Sherwood, AR (US)

Correspondence Address: McTavish Patent Firm, 429 Birchwood Courts, Birchwood, MN, 55110, US

(73) Assignee: The Board Trustees of the University of Arkansas, Little Rock, AR (US)

(21) Appl. No.: 10/992,916

(22) Filed: Nov. 19, 2004

(51) Int. Cl. C12Q 1/04; C07D 215/36

(52) U.S. Cl. 435/34; 546/156

(57) ABSTRACT

The invention provides methods of diagnosing and imaging bacterial infections in mammals using ¹⁸F-labeled fluoroquinolone antibiotics and detecting the fluorine-18. Fluorine-18 can be detected and imaged by positron emission tomography. The invention also provides methods of detecting bacteria in other settings, and of treating a bacterial infection and monitoring treatment of a bacterial infection.

### BACKGROUND

A radiopharmaceutical, technetium-99m (^{99m}Tc) INFECTON, has been developed for imaging areas of bacterial infection and diagnosing bacterial infection. (Vinjamuri, S. et al. 1996, Lancet 347:233-235; Britton, K. E. et al. 1997, Eur. J. Nucl. Med. 24:553-556; Hall, A. V. et al., 1998, J. Clin. Pathol. 51:215-219; Britton, K. E. 2002, J. Clin. Pathol. 55:817-823.) ^{99m}Tc INFECTON includes the fluoroquinolone antibiotic ciprofloxacin chelated to a ^{99m}Tc radionuclide.

^{99m}Tc INFECTON was used to diagnose bacterial infection on 90 patients thought to be suffering from infection. (Hall, A. V. et al., 1998, J. Clin. Pathol. 51:215-219.) Hall et al. report 98 images were produced. Forty-one were positive including three false positive. Fifty-seven were negative, including 16 false negative. A high false negative rate was obtained with osteomyelitis patients and pneumonia patients. Patients suspected of having septic arthritis produced two of the three false positives, suggesting that inflammation may lead to false positives. (Hall, A. V. et al., 1998, J. Clin. Pathol. 51:215-219.)

Another method of imaging infections is the use of Indium-111-labeled leukocytes or ^{99m}Tc-HMPAO-labeled leukocytes. (Datz, F. L. 1994, Sem. Nucl. Med. 24:92-109; Peters, A. M. et al. 1986, Lancet 2:946-949.) In this method, leukocytes or a subset of leukocytes (e.g., neutrophils) are separated from a patient's blood, labeled with the radioisotope, and then reinjected into the patient. Their location is then visualized by autoradiography or by imaging with an Anger-type gamma camera. This can identify sites of inflammation, but is not specific for bacterial infections. It is also rather labor intensive.

New methods of diagnosing suspected bacterial infection, and of determining the location of infected tissues and the nature of infection so the infection can be more effectively treated are needed.

### SUMMARY

A new method of detecting and imaging the location of bacteria is provided. The method involves contacting bacteria (e.g., in a mammal) with a fluoroquinolone antibiotic, such as ciprofloxacin, labeled with fluorine-18. Fluorine is an element present in the fluoroquinolone antibiotics. Thus, the one or more fluorine atoms in the antibiotics can be exchanged for the F-18 isotope without a change in the chemical characteristics of the molecule. F-18 is a positron-emitting radioisotope. It can be detected and visualized in a mammal by positron emission tomography (PET) or by autoradiography, or by measurement with radiation detection devices, such as survey meters or well counters.

Ciprofloxacin and other fluoroquinolone antibiotics inhibit DNA gyrase and other topoisomerases in bacteria. Importantly, most bacteria that are resistant to the fluoroquinolones still bind them. Thus, the F-18-labeled fluoroquinolones of the present invention can be used to detect and image antibiotic-resistant bacteria as well as antibiotic-sensitive bacteria. The quantitative nature of the binding and of the PET imaging methodology allows quantitative detection of bacteria. Some other methods of imaging infection (e.g., use of radiolabeled leukocytes) detect inflammation and do not directly detect bacteria. In contrast, F-18-labeled fluoroquinolones directly detect bacteria and thus distinguish between bacterial infection and other causes of inflammation, including viral infection and post-surgical healing.

The invention provides a method of detecting bacteria involving contacting a sample suspected of containing bacteria with a fluorine-18-labeled fluoroquinolone antibiotic, and detecting the fluorine-18-labeled antibiotic by detecting emission of positrons from fluorine-18. The method is non-invasive and can involve either counting and/or imaging the sample.

Another embodiment of the invention provides a method of diagnosing a bacterial infection in a mammal involving: administering a fluorine-18-labeled fluoroquinolone antibiotic to the mammal; and determining location of the fluorine-18 in the mammal; wherein the location of the fluorine-18 in the mammal is used to diagnose the presence or absence of bacterial infection in the mammal and the location of any bacterial infection. Determining the location of the fluorine-18 in the mammal can be done either qualitatively or quantitatively.

Another embodiment of the invention provides a method of treating a bacterial infection in a mammal involving: (a) administering a fluorine-18-labeled fluoroquinolone antibiotic to the mammal; (b) determining location of the fluorine-18 in the mammal; (c) diagnosing location of tissue infected with bacteria from the location of the fluorine-18 mammal in the mammal; and (d) treating the bacterial infection. Steps (a) through (d) can optionally be repeated after treatment to determine efficacy of treatment and/or quantitative changes between the measurements (such as partial response to treatment and the need to continue treatment).

Another embodiment of the invention provides a method of monitoring treatment of a bacterial infection in a mammal undergoing treatment, the method involving: (a) administering a fluorine-18-labeled fluoroquinolone antibiotic to the mammal undergoing treatment for the bacterial infection; (b) determining location of the fluorine-18 in the mammal; and (c) diagnosing location of tissue infected with bacteria from the location of fluorine-18 in the mammal.

### DETAILED DESCRIPTION

Definitions:

A “fluoroquinolone antibiotic” refers to an antibiotic with a 4-quinolone core, having structure I 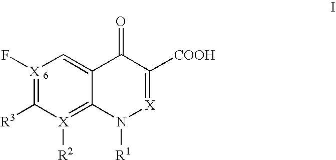
and having antibiotic activity. Each X is independently C or N. R¹, R², and R³ can be any suitable substituents that result in a compound with antibiotic activity.

A “fluorine-18-labeled fluoroquinolone antibiotic” is any fluoroquinolone antibiotic that includes a fluorine-18 atom. All fluoroquinolones contain a fluorine at position 6 of the 4-quinolone ring. Often, this fluorine atom is a fluorine-18 atom in the fluorine-18-labeled fluoroquinolone antibiotics of the invention. But many fluoroquinolone antibiotics contain other fluorine atoms, and any of these can be the F-18 in the labeled antibiotics in addition to or in place of the fluorine atom at position 6.

Description:

The method of detecting bacteria with an F-18-labeled fluoroquinolone antibiotic can detect bacteria in a living mammal, e.g., a human, to diagnose infection, monitor treatment of an infection, or as part of a method of treating infection. In this embodiment, the F-18 antibiotic is typically injected into the mammal, allowed time to circulate and bind to any bacterial infection (e.g., an hour), and then counted or visualized. The method can also be used to detect bacteria in food. The method can also be used to detect bacteria on inanimate surfaces or in containers. The method can be used to detect bacteria in samples suspected of containing bacteria used in bioterrorism, such as suspicious powders in envelopes.

When the F-18 labeled antibiotic is administered to a mammal it can be administered by any suitable route, including orally, intravenously, intramuscularly, subcutaneously, or intraperitoneally. Intravenous injection is a particularly preferred route of administration.

In particular embodiments of the method of detecting bacteria, after the F-18-labeled antibiotic is contacted with the sample, the method includes washing unbound fluoroquinolone antibiotic containing F-18 from the sample. This is particularly done when the sample is not a mammal, e.g., where the sample is food or an inanimate surface being tested for bacteria. Washing is preferably done with a liquid in which the antibiotic readily dissolves. Washing may involve blowing air or other gas over the sample to remove unbound antibiotic in the gas.

In the methods of the invention, the steps of detecting the fluorine-18-labeled antibiotic or determining the location of fluorine-18 can be done by positron emission tomography. In particular embodiments, the detecting or localizing can involve use of a geiger counter or other survey meter-type device, or other radiation detection instrument or method, such as, but not limited to, autoradiography, a well counter, a dose calibrator, or an uptake probe. In particular embodiments, the detecting or localizing can involve use of a geiger counter, well counter, dose calibrator, or uptake probe.

The fluoroquinolone antibiotics bind to almost all species of bacteria, including most bacteria that are resistant to the fluoroquinolone antibiotic. Thus, the methods of the invention can be used to detect any type of bacteria and to diagnose or monitor infection by any type of bacteria. The method is suitable, for instance, to detect mycobacteria, including mycobacteria responsible for tuberculosis and atypical tuberculosis (e.g., Mycobacterium tuberculosis).

In particular embodiments of the invention, the fluoroquinolone antibiotic is ciprofloxacin. In other embodiments, the fluoroquinolone antibiotic is not ciprofloxacin.

In particular embodiments, the F-18-labeled fluoroquinolone antibiotic is alatrofloxacin, clinafloxicin, enoxacin, fleroxacin, flumequine, levofloxacin, lomefloxacin, norfloxacin, ofloxacin, pefloxacin, rufloxacin, sparfloxacin, tosufloxacin, or trovafloxacin. The fluoroquinolone antibiotic may be any fluoroquinolone antibiotic that exists today or is developed in the future.

In other specific embodiments, the fluoroquinolone antibiotic containing fluorine-18 is alatrofloxacin, clinafloxicin, enoxacin, fleroxacin, flumequine, levofloxacin, norfloxacin, ofloxacin, pefloxacin, rufloxacin, sparfloxacin, or tosufloxacin.

In specific embodiments, the fluoroquinolone antibiotic containing fluorine-18 is not ciprofloxacin, trovafloxacin, or lomefloxacin.

In particular embodiments, the fluoroquinolone antibiotic containing fluorine-18 is a compound of formula II

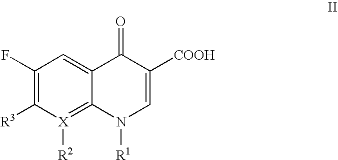

wherein

X is N or C;

R¹ is H, (C₁-C₆)alkyl; or a 3- to 8-membered saturated or unsaturated ring of carbon atoms and optionally N, O, or S atoms;

R² is H or halo;

or R¹ and R² together are a 3- to 5-membered linear saturated or unsaturated chain of carbon atoms and optionally N, O, or S atoms; and

R³ is H, (C₁-C₆)alkyl, or a 3- to 8-membered saturated or unsaturated ring of carbon atoms and optionally N, O, or S atoms;

wherein the (C₁-C₆)alkyl of R¹ and R³, the 3- to 8-membered ring of R¹ and R³ and the 3- to 5-membered linear chain of R¹ and R² together are each optionally substituted with halo, (C₁-C₃)alkyl, OH, (C₁-C₃)alkoxy, —N(R⁴)₂, or phenyl; where each R⁴ is independently H, (C₁-C₆)alkyl, or phenyl.

In other embodiments, the fluorine-18-labeled fluoroquinolone antibiotic is a compound of formula II

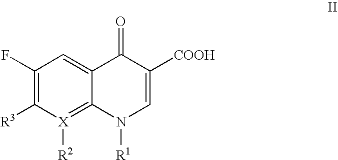

wherein

X is N or C;

R¹ is H, (C₁-C₆)alkyl; or a 3- to 8-membered saturated or unsaturated ring of carbon atoms and optionally N or O atoms, wherein the alkyl and 3- to 8-membered ring are optionally substituted with halo;

R² is H or halo;

or R¹ and R² together are a 3- to 5-membered linear saturated or unsaturated chain of carbon atoms and optionally N or O atoms; and

R³ is H, or a 5- to 6-membered saturated or unsaturated heteroalkyl ring containing 1 or 2 N atoms, optionally substituted with (C₁-C₃)alkyl or —NH₂.

In particular embodiments of the method of detecting bacteria, the sample is a mammal and the step of contacting the sample with a fluorine-18-labeled fluoroquinolone antibiotic involves intravenously injecting the mammal with the fluoroquinolone antibiotic containing fluorine-18.

In some embodiments of the method of treating a bacterial infection in a mammal, the step of treating the bacterial infection after diagnosing location of tissue infected with bacteria includes surgically removing some or all of the tissue infected with bacteria.

In some embodiments, the step of treating the bacterial infection includes locally administering antibiotics to the tissue infected with bacteria.

In some embodiments, the step of treating the bacterial infection includes systemically administering antibiotics to the mammal.

In the methods of detecting bacteria, diagnosing a bacterial infection, treating a bacterial infection, and monitoring treatment of a bacterial infection, the methods can include determining density of fluorine-18 in the tissue infected with bacteria. That is typically done by positron emission tomography.

¹⁸F-labeled fluoroquinolone antibiotics can be prepared most simply by an exchange reaction with heating in the presence of ¹⁸F-fluoride, as described below in Example 2. In some cases, such as with ciprofloxacin, the exchange reaction may not be successful (Langer, O. et al., 2003, Nucl. Med. Biol. 30:285-291). In those cases, an antibiotic precursor compound can be subject to the exchange reaction, and then a one-step addition reaction can be performed on the ¹⁸F-labeled precursor to synthesize the ¹⁸F-labeled fluoroquinolone antibiotic product. An example of this procedure is provided in Example 1 below. The half-life of ¹⁸F is 110 minutes, so it is important that the ¹⁸F-labeled fluoroquinolone antibiotic be prepared in a short time after generation of the ¹⁸F and that the ¹⁸F-labeled antibiotic be used fairly quickly after it is prepared.

The invention will now be illustrated by the following non-limiting examples.

### EXAMPLES

### Example 1

### Imaging with [¹⁸F] Ciprofloxacin

Methods:

¹⁸F synthesis. Aqueous [¹⁸F]fluoride is produced in a General Electric PETTRACE cyclotron (General Electric, USA) via the ¹⁸O(p,n)¹⁸F nuclear reaction by irradiation of a 1.5 ml water target containing 95.9% enriched [¹⁸O]water (HYOX¹⁸, Rotem Industries, Beer Sheva, Israel) with a 16.5 MeV proton beam. ¹⁸O is converted to ¹⁸F by proton bombardment. Typically, a 60 minute irradiation with a beam current of 35 μA yields about 60 GBq of [¹⁸F]fluoride.

7-chloro-1 -cyclopropyl-6-[¹⁸F]fluoro-1,4-dihydro-4-oxoquinoline-3 -carboxylic acid ([¹⁸F]III) synthesis. (Langer, O. et al. 2003, Nucl. Med. Biol. 30:285-291.) Aqueous [¹⁸F]fluoride ion is recovered from the cyclotron target into a 3 ml vial containing acetonitrile (0.5 ml), KRYPTOFIX 2.2.2 (4,7,13,16,21,24-hexaoxa-1,10-diazabicyclo[8,8,8]hexacosane, 12.0 mg, 32.7 μmol), and potassium carbonate (4.0 mg, 28.9 μmol). The mixture is brought to dryness at 180° C. under a stream of nitrogen with repeated addition of acetonitrile (2×0.5 ml), which affords the K[¹⁸F]F-K₂₂₂ complex as a yellow semi-solid residue. To this residue 7-chloro-1 -cyclopropyl-6-fluoro-1,4-dihydro-4-oxoquinoline-3-carboxylic acid (III, 6.0-7.0 mg, 21.3-24.9 μmol) dissolved in DMSO (0.3 ml) is added and the resulting solution stirred at 180° C. for 40 minutes. An aliquot of the reaction mixture is analyzed by HPLC and TLC.

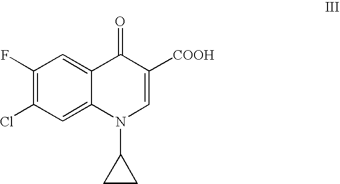

[¹⁸F]ciprofloxacin synthesis. ([¹⁸F]IV) (Langer, O. et al. 2003, Nucl. Med Biol. 30:285-291.) [¹⁸F]ciprofloxacin is synthesized by the addition of piperazine to [¹⁸F]III. After the exchange reaction, the reaction mixture is cooled in an ethanol bath for 2 minutes. Then a mixture of trimethylborate (20 μl, 178 μmol) and acetic acid (20 μl, 350 μmol) in DMSO (0.1 ml) is added and the solution stirred at room temperature for 2 minutes. Then piperazine (20-25 mg, 232-290 μmol) dissolved in DMSO is added and the reaction stirred at 180° C. for 40 minutes. The vial is then cooled in an ethanol bath for 2 minutes and 0.9 ml of mobile phase for semipreparative HPLC is added.

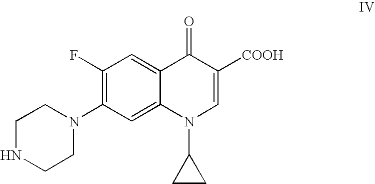

The product is purified by isocratic elution with 10 mM aqueous phosphoric acid and absolute ethanol (88/12 v/v) on a C18 column. Product can be detected by monitoring uv absorption or radioactivity. The product fraction is loaded on a strong cation exchange cartridge (ISOLUTE 100 mg SCX, International Sorbent Technology Ltd., Hengoed, UK). Prior to use the cartridge is prewashed with absolute ethanol and the HPLC mobile phase. After loading the product, the cartridge is dried with an air stream and eluted with 0.1 M NaOH (3.0 ml into a sterile 11 ml vial containing 0.20 M phosphate buffer (5.0 ml, pH 3.0-3.5).

The product mixture is then homogenized and filtered through a 0.22 μm filter into a sterile vial.

[¹⁸F]ciprofloxacin administration to human subjects suspected of having bacterial infection and PET monitoring of the localization of [¹⁸F] in the subjects. Healthy subjects are injected intravenously with 700 to 1400 MBq of [¹⁸F]ciprofloxacin. In order to correct for the attenuation of photons by tissue, a transmission scan with two 400-MBq pin sources or from CT data (if the scan is acquired on a PET/CT scanner) is recorded after obtaining the emission scan. Thirty to sixty minutes after injection of [¹⁸F]ciprofloxacin, data are obtained by PET scan of the regions of concern (such as the whole body or a specific area such as the hip or chest, depending on the clinical need) . Each subject is positioned supine on the imaging bed. The data on healthy subjects establish the appearance of the distribution of [¹⁸F]ciprofloxacin in uninfected patients.

The same protocol is used on patients diagnosed by other criteria with bacterial bone infections, bacterial wound infections, bacterial respiratory infections, and other bacterial infections.

Results:

It is determined that the PET scan results of patients with the various bacterial infections tested are distinguishable from the PET scan results of healthy volunteers, and allow the diagnosis of bacterial infection and the diagnosis of the location of infected tissue.

### Example 2

### Imaging with [¹⁸F]Fleroxacin

Methods:

[¹⁸F]Fluoride is prepared as in Example 1. Aqueous [¹⁸F]fluoride ion is recovered from the cyclotron target into a 3 ml vial containing acetonitrile (0.5 ml), KRYPTOFIX 2.2.2 (4,7,13,16,21,24-hexaoxa-1,10-diazabicyclo[8,8,8]hexacosane, 12.0 mg, 32.7 μmol), and potassium carbonate (4.0 mg, 28.9 μmol). The mixture is brought to dryness at 180° C. under a stream of nitrogen with repeated addition of acetonitrile (2×0.5 ml), which affords the K[¹⁸F]F-K₂₂₂ complex as a yellow semi-solid residue.

Fleroxacin (1 mg) in 0.5 ml DMSO is added to is added to the dry K[¹⁸F]F-K₂₂₂ complex and heated at 150° C. for approximately 1 hour. The [¹⁸F]fleroxacin product is then purified by HPLC with monitoring of the eluate of the column for uv absorption and radioactivity, as described in Example 1.

Healthy subjects and subjects confirmed by conventional diagnostic methods to have bacterial infections of various types are injected intravenously with 700 to 1400 MBq of [¹⁸F]fleroxacin. After 30 to 60 minutes, the subjects are subjected to PET scan imaging as described in Example 1.

Results:

It is determined that the PET scan results of patients with the various bacterial infections tested are distinguishable from the PET scan results of healthy volunteers, and allow the diagnosis of bacterial infection and the diagnosis of the location of infected tissue.

What is claimed is:

1. A method of detecting bacteria comprising:
contacting a sample suspected of containing bacteria with a fluorine-18-labeled fluoroquinolone antibiotic; and
detecting the fluorine-18-labeled antibiotic by detecting emission of positrons from fluorine-18.

2. The method of claim 1 further comprising between the contacting and detecting steps:
washing unbound fluorine-18-labeled fluoroquinolone antibiotic from the sample.

3. The method of claim 1 wherein the detecting emission of positrons from fluorine-18 is by positron emission tomography.

4. The method of claim 1 wherein the detecting emission of positrons from fluorine-18 is performed with a geiger counter, well counter, dose calibrator, or uptake probe.

5. The method of claim 1 wherein the fluorine-18-labeled fluoroquinolone antibiotic is ciprofloxacin.

6. The method of claim 1 wherein the fluorine-18-labeled fluoroquinolone antibiotic is not ciprofloxacin.

7. The method of claim 1 wherein the fluorine-18-labeled fluoroquinolone antibiotic is alatrofloxacin, clinafloxicin, enoxacin, fleroxacin, flumequine, levofloxacin, lomefloxacin, norfloxacin, ofloxacin, pefloxacin, rufloxacin, sparfloxacin, tosufloxacin, or trovafloxacin.

8. The method of claim 1 wherein the fluorine-18-labeled fluoroquinolone antibiotic is alatrofloxacin, clinafloxicin, enoxacin, fleroxacin, flumequine, levofloxacin, norfloxacin, ofloxacin, pefloxacin, rufloxacin, sparfloxacin, or tosufloxacin.

9. The method of claim 1 wherein the fluorine-18-labeled fluoroquinolone antibiotic is a compound of formula II 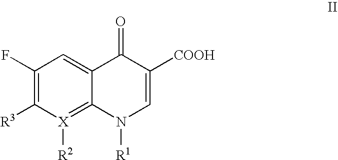
wherein
X is N or C;
R¹ is H, (C₁-C₆)alkyl; or a 3- to 8-membered saturated or unsaturated ring of carbon atoms and optionally N, O, or S atoms;
R² is H or halo;
or R¹ and R² together are a 3- to 5-membered linear saturated or unsaturated chain of carbon atoms and optionally N, O, or S atoms; and
R³ is H, (C₁-C₆)alkyl, or a 3- to 8-membered saturated or unsaturated ring of carbon atoms and optionally N, O, or S atoms;
wherein the (C₁-C₆)alkyl of R¹ and R³, the 3- to 8-membered ring of R¹ and R³ and the 3- to 5-membered linear chain of R¹ and R² together are each optionally substituted with halo, (C₁-C₃)alkyl, OH, (C₁-C₃)alkoxy, —N(R⁴)₂, or phenyl; where each R⁴ is independently H, (C₁-C₆)alkyl, or phenyl.

10. The method of claim 9 wherein the fluorine-18-labeled fluoroquinolone antibiotic is a compound of formula II 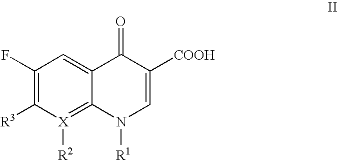
wherein
X is N or C;
R¹ is H, (C₁-C₆)alkyl; or a 3- to 8-membered saturated or unsaturated ring of carbon atoms and optionally N or O atoms, wherein the alkyl and 3- to 8-membered ring are optionally substituted with halo;
R²is H or halo;
or R¹ and R² together are a 3- to 5-membered linear saturated or unsaturated chain of carbon atoms and optionally N or O atoms; and
R³ is H, or a 5- to 6-membered saturated or unsaturated heteroalkyl ring containing 1 or 2 N atoms, optionally substituted with (C₁-C₃)alkyl or —NH₂.

11. The method of claim 1 wherein the sample is a mammal and the step of contacting the sample with a fluorine-18-labeled fluoroquinolone antibiotic comprises intravenously injecting the mammal with the fluorine-18-labeled fluoroquinolone antibiotic.

12. The method of claim 1 wherein the sample is a food sample.

13. A method of diagnosing a bacterial infection in a mammal comprising:
administering a fluorine-18-labeled fluoroquinolone antibiotic to the mammal; and
determining location of the fluorine-18 in the mammal;
wherein the location of the fluorine-18 in the mammal is used to diagnose the presence or absence of bacterial infection in the mammal and the location of any bacterial infection.

14. The method of claim 13 wherein the administering step comprises orally, intravenously, intramuscularly, subcutaneously, or intraperitoneally administering the fluorine-18-labeled fluoroquinolone antibiotic to the mammal.

15. The method of claim 13 wherein the step of determining the location of the fluorine-18 in the mammal comprises imaging the mammal by positron emission tomography.

16. A method of treating a bacterial infection in a mammal comprising:
administering a fluorine-18-labeled fluoroquinolone antibiotic to the mammal;
determining location of the fluorine-18 in the mammal;
diagnosing location of tissue infected with bacteria from the location of the fluorine-18 mammal in the mammal;
and treating the bacterial infection.

17. The method of claim 16 wherein the step of treating the bacterial infection comprises surgically removing some or all of the tissue infected with bacteria.

18. The method of claim 16 wherein the step of treating the bacterial infection comprises locally administering antibiotics to the tissue infected with bacteria.

19. A method of monitoring treatment of a bacterial infection in a mammal undergoing such treatment comprising:
administering a fluorine-18-labeled fluoroquinolone antibiotic to the mammal undergoing treatment for a bacterial infection;
determining location of the fluorine-18 in the mammal; and
diagnosing location of tissue infected with bacteria from the location of fluorine-18 in the mammal.

20. The method of claim 19 further comprising determining density of fluorine-18 in the tissue infected with bacteria.
